(19) United States
(12) Patent Application Publication
(10) Pub. No.: US 2018/0013999 A1
(43) Pub. Date: Jan. 11, 2018

(54) ENDOSCOPE PROCESSOR

(71) Applicant: OLYMPUS CORPORATION, Tokyo (JP)

(72) Inventors: Soichiro KOSHIKA, Tokyo (JP); Masanori SUMIYOSHI, Tokyo (JP)

(73) Assignee: OLYMPUS CORPORATION, Tokyo (JP)

(21) Appl. No.: 15/642,445

(22) Filed: Jul. 6, 2017

(30) Foreign Application Priority Data
Jul. 7, 2016 (JP) 2016-135453

(51) Int. Cl. H04N 9/64; H04N 9/07; A61B 1/04

(52) U.S. Cl. CPC H04N 9/646; A61B 1/04; H04N 9/07

(57) ABSTRACT

An endoscope processor includes: an image generation portion that generates a picked-up image of a subject, an image of which is picked up by an endoscope; a correction information acquisition portion that acquires correction information corresponding to magnification chromatic aberration of the endoscope from a scope memory in the endoscope; and an image correction portion that corrects the magnification chromatic aberration in the picked-up image based on the correction information.

### CROSS REFERENCE TO RELATED APPLICATION

This application claims the benefit of Japanese Application No. 2016-135453 filed in Japan on Jul. 7, 2016, the contents of which are incorporated herein by this reference.

### BACK GROUND OF THE INVENTION

1. Field of the Invention

The present invention relates to an endoscope processor of an endoscope apparatus.

2. Description of the Related Art

Conventionally, endoscope apparatuses, which are configured to apply illumination light from a distal end portion of an insertion portion of an endoscope to a subject, receive return light from the subject, and pick up an image of the subject, have been used. In such endoscope apparatuses, there is a case where color shift occurs in the picked-up image due to chromatic aberration of an optical system provided at the distal end portion of the insertion portion, and correction of magnification chromatic aberration is performed by image processing and the like.

Japanese Patent No. 5490331, for example, discloses a scanning endoscope apparatus that detects an aberration amount corresponding to a predetermined image height based on a predetermined aberration diagram, performs image processing for reducing or expanding each of red and blue images according to the detected aberration amount, and corrects the magnification chromatic aberration.

### SUMMARY OF THE INVENTION

An endoscope processor according to one aspect of the present invention includes an image generation portion that generates a picked-up image of a subject, and image of which is picked up by an endoscope, a correction information acquisition portion that acquires correction information corresponding to magnification chromatic aberration of the endoscope from a scope memory in the endoscope, and an image correction portion that corrects the magnification chromatic aberration in the picked-up image based on the correction information.

### BRIEF DESCRIPTION OF THE DRAWINGS

FIG. 1 is an explanatory diagram for describing an exemplary configuration of an endoscope apparatus according to an embodiment of the present invention.

FIG. 2A is an explanatory diagram for describing an exemplary configuration of an illumination portion of the endoscope apparatus according to the embodiment of the present invention.

FIG. 2B is a cross-sectional view showing an exemplary configuration of an actuator of the endoscope apparatus according to the embodiment of the present invention.

FIG. 3A is an explanatory diagram for describing a spiral-shaped scanning path of the endoscope apparatus according to the embodiment of the present invention.

FIG. 3B is an explanatory diagram for describing a spiral-shaped scanning path of the endoscope apparatus according to the embodiment of the present invention.

FIG. 4 is a chart showing an example of an association table of the endoscope apparatus according to the embodiment of the present invention.

FIG. 5 is a chart showing an example of a correction table of the endoscope apparatus according to the embodiment of the present invention.

FIG. 6 illustrates an example of a measurement chart of the endoscope apparatus according to the embodiment of the present invention.

FIG. 7A is an explanatory diagram for describing an exemplary configuration of the illumination portion of the endoscope apparatus according to the embodiment of the present invention.

FIG. 7B is an explanatory diagram for describing magnification chromatic aberration in a picked-up image obtained by the endoscope apparatus according to the embodiment of the present invention.

FIG. 7C is a chart showing an example of a correction table of the endoscope apparatus according to the embodiment of the present invention.

FIG. 8A is an explanatory diagram for describing an exemplary configuration of the illumination portion of the endoscope apparatus according to the embodiment of the present invention.

FIG. 8B is an explanatory diagram for describing the magnification chromatic aberration in the picked-up image obtained by the endoscope apparatus according to the embodiment of the present invention.

FIG. 8C is a chart showing an example of a correction table of the endoscope apparatus according to the embodiment of the present invention.

FIG. 9A is an explanatory diagram for describing an exemplary configuration of the illumination portion of the endoscope apparatus according to the embodiment of the present invention.

FIG. 9B is an explanatory diagram for describing the magnification chromatic aberration in the picked-up image obtained by the endoscope apparatus according to the embodiment of the present invention.

FIG. 9C is a chart showing an example of the correction table of the endoscope apparatus according to the embodiment of the present invention.

FIG. 10A is an explanatory diagram for describing an exemplary configuration of the illumination portion of the endoscope apparatus according to the embodiment of the present invention.

FIG. 10B is an explanatory diagram for describing the magnification chromatic aberration in the picked-up image obtained by the endoscope apparatus according to the embodiment of the present invention.

FIG. 10C is a chart showing an example of the correction table of the endoscope apparatus according to the embodiment of the present invention.

FIG. 11 is a flowchart showing an example of a flow of key information setting processing of the endoscope apparatus according to the embodiment of the present invention.

FIG. 12A is a graph showing a relation between a pixel position and a signal level in the picked-up image obtained by the endoscope apparatus according to the embodiment of the present invention.

FIG. 12B is a graph showing a relation between a pixel position and a signal level in a picked-up image obtained by the endoscope apparatus according to the embodiment of the present invention.

FIG. 13 is a flowchart showing an example of a flow of image correction processing in the endoscope apparatus according to the embodiment of the present invention.

### DETAILED DESCRIPTION OF THE PREFERRED EMBODIMENTS

Hereinafter, an embodiment of the present invention will be described with reference to drawings.

### (Configuration)

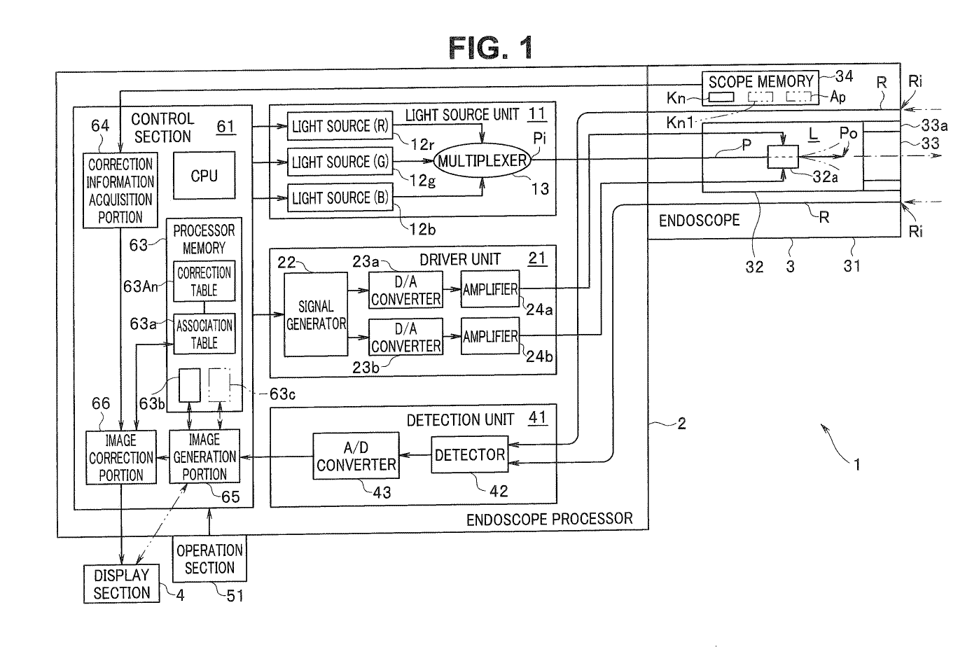

FIG. 1 is a block diagram illustrating an exemplary configuration of an endoscope apparatus 1 according to the embodiment of the present invention.

The endoscope apparatus 1 is a scanning endoscope apparatus, and includes an endoscope processor 2, an endoscope 3, and a display section 4, as shown in FIG. 1. The endoscope 3 and the display section 4 are detachably connected to the endoscope processor 2.

The endoscope processor 2 includes a light source unit 11, a driver unit 21, a detection unit 41, an operation section 51, and a control section 61.

The light source unit 11 is configured to generate red laser light, green laser light, and blue laser light based on a control signal inputted from the control section 61 to be described later, and enable the respective laser light to enter an incident end Pi of an illumination optical fiber P. The light source unit 11 includes a red laser light source 12r, a green laser light source 12g, a blue laser light source 12b, and a multiplexer 13. The red, green, and blue laser light sources 12r, 12g, and 12b are connected to the multiplexer 13. The light source unit 11 is connected to the illumination optical fiber P. The light source unit 11 outputs the red laser light, the green laser light, and the blue laser light sequentially as illumination light to the illumination optical fiber P.

The illumination optical fiber P includes an incident end Pi on which the illumination light is incident, and an emission end Po from which the illumination light is emitted to a subject, and is configured to be capable of guiding light from the incident end Pi to the emission end Po. The illumination optical fiber P emits the illumination light, which is inputted from the light source unit 11, from the distal end of the insertion portion 31 of the endoscope 3 to the subject.

The driver unit 21 is a circuit that drives an actuator 32a of the endoscope 3 and causes the emission end Po of the illumination optical fiber P to swing. The driver unit 21 includes a signal generator 22, D/A converters 23a, 23b, and amplifiers 24a, 24b. FIG. 1 schematically shows a state where the emission end Po swings, by the two-dot-chain lines.

The signal generator 22 generates drive signals DX, DY for driving the actuator 32a based on the control signals inputted from the control section 61 and outputs the generated drive signals to the D/A converters 23a, 23b.

The drive signal DX is outputted so as to enable the emission end Po of the illumination optical fiber P to swing in an X-axis direction as described later. The drive signal DX is defined by an expression (1) below, for example. In the expression (1), X(t) represents a signal level of the drive signal DX at a time t, AX represents an amplitude value that is independent of the time t, and G(t) represents a predetermined function for modulating a sine-wave sin (27 πft).

X(t)=AX×G(t)×sin (2 πft) (1)

The drive signal DY is outputted so as to enable the emission end Po of the illumination optical fiber P to swing in a Y-axis direction as described later. The drive signal DY is defined by an expression (2) below, for example. In the expression (2), Y(t) represents a signal level of the drive signal DY at the time t, AY represents an amplitude value that is independent of the time t, G(t) represents a predetermined function for modulating a sine-wave sin(2 πft+φ), and φ represents a phase.

Y(t)=AY×G(t)×sin (2 πft+φ) (2)

The D/A converters 23a, 23b convert the drive signals DX, DY inputted from the signal generator 22 from digital signals into analog signals, and output the analog signals to the amplifiers 24a, 24b.

The amplifiers 24a, 24b amplify the drive signals DX, DY inputted from the D/A converters 23a, 23b, and output the amplified drive signals DX, DY to the actuator 32a.

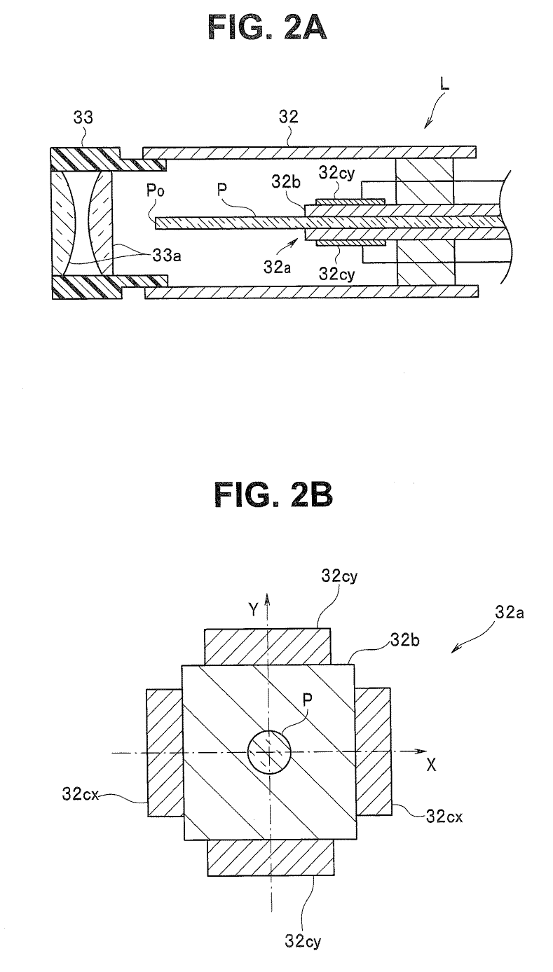

FIG. 2A is an explanatory diagram for describing an exemplary configuration of an illumination portion L of the endoscope apparatus 1 according to the embodiment of the present invention. FIG. 2B is a cross-sectional view showing an exemplary configuration of the actuator 32a of the endoscope apparatus 1 according to the embodiment of the present invention. In FIG. 2B, the X-axis direction is the direction perpendicular to the longitudinal axis direction of the illumination optical fiber P, and the Y-axis direction is the direction perpendicular to the longitudinal axis of the illumination optical fiber P and the X-axis direction.

The endoscope 3 is inserted into a subject and configured to apply the light emitted from the light source unit 11 to the subject, and to be capable of picking up an image of the return light from the subject. The endoscope 3 includes an insertion portion 31, a protection pipe 32 and a scope barrel 33 that constitute the illumination portion L, a light-receiving portion Ri, and a scope memory 34.

The insertion portion 31 is formed in an elongated shape, and insertable into a body of a subject. As shown in FIG. 2A, at the distal end of the insertion portion 31, the protection pipe 32 and the scope barrel 33 are provided.

The protection pipe 32 is made of metal, for example. The protection pipe 32 is formed in a cylindrical shape. The protection pipe 32 houses inside thereof the actuator 32a and the emission end Po.

The actuator 32a causes the emission end Po to swing, and is capable of moving the application position of the illumination light along a predetermined scanning path. The predetermined scanning path is a spiral-shaped scanning path, for example. As shown in FIG. 2B, the actuator 32a includes a ferrule 32b, and piezoelectric elements 32cx, 32cy.

The ferrule 32b is made of zirconia (ceramic), for example. The ferrule 32b is provided in the vicinity of the emission end Po so as to be capable of swinging the emission end Po.

The piezoelectric elements 32cx, 32cy vibrate according to the drive signals DX, DY inputted from the driver unit 21 to cause the emission end Po to swing. The emission end Po is caused to swing in the X-axis direction by the piezoelectric element 32cx, and caused to swing in the Y-axis direction by the piezoelectric element 32cy (FIG. 2B).

The scope barrel 33 is made of resin or the like, for example. The scope barrel 33 is formed in a cylindrical shape and holds on the inner circumferential side thereof an optical system 33a. The scope barrel 33 is attached to the distal end of the protection pipe 32 and fixed thereto with adhesive or the like.

The optical system 33a is configured such that the illumination light emitted from the emission end Po can be applied to the subject. When the scope barrel 33 is fixed to the protection pipe 32, the attaching position of the optical system 33a is also determined. Note that the optical system 33a is configured by two plano-convex lenses in FIG. 2A, but the configuration of the optical system 33a is not limited thereto.

The light-receiving portion Ri is provided at the distal end of the insertion portion 31, and receives the return light from the subject. The received return light from the subject is outputted to the detection unit 41 in the endoscope processor 2 through a light-receiving optical fiber R.

The scope memory 34 is configured by a memory such as a nonvolatile memory and stores key information Kn.

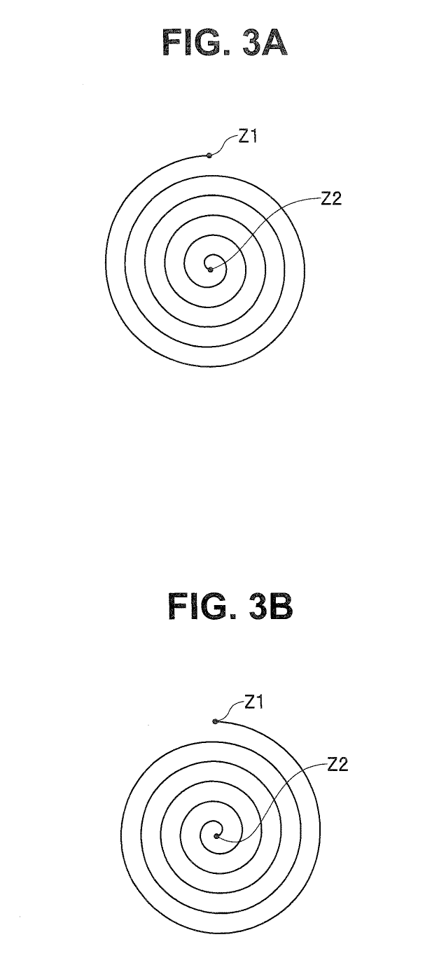

FIG. 3A is an explanatory diagram for describing a spiral-shaped scanning path of the endoscope apparatus 1 according to the embodiment of the present invention. FIG. 3B is an explanatory diagram for describing a spiral-shaped scanning path of the endoscope apparatus 1 according to the embodiment of the present invention.

When the driver unit 21 outputs the drive signals DX, DY while increasing the level of the signals, the illumination optical fiber P is swung by the actuator 32a and the application position of the illumination optical fiber P moves along the spiral-shaped scanning path that gradually gets away from the center, as shown from Z2 to Z1 in FIG. 3A. After that, when the driver unit 21 outputs the drive signals DX, DY while decreasing the level of the signals, the application position of the illumination optical fiber P moves along the spiral-shaped scanning path that gradually gets close to the center, as shown from Z1 to Z2 in FIG. 3B. According to such a configuration, the red laser light, the green laser light, and the blue laser light that are sequentially generated by the light source unit 11 are applied spirally to the subject. The return light from the subject is received by the light-receiving portion Ri and the subject is scanned spirally.

With reference back to FIG. 1, the detection unit 41 is a circuit that detects the return light from the subject and outputs a detection signal according to the return light to the control section 61. The detection unit 41 includes a detector 42, and an A/D converter 43.

The detector 42 includes a photoelectric conversion device, and converts the return light from the subject, which is inputted from the light-receiving portion Ri through the light-receiving optical fiber R, into a detection signal indicating red, green and blue colors, to output the detection signal to the A/D converter 43.

The A/D converter 43 converts the detection signal inputted from the detector 42 into a digital signal, to output the digital signal to the control section 61.

The operation section 51 is connected to the control section 61 and configured to be capable of outputting an instruction input by a user to the control section 61.

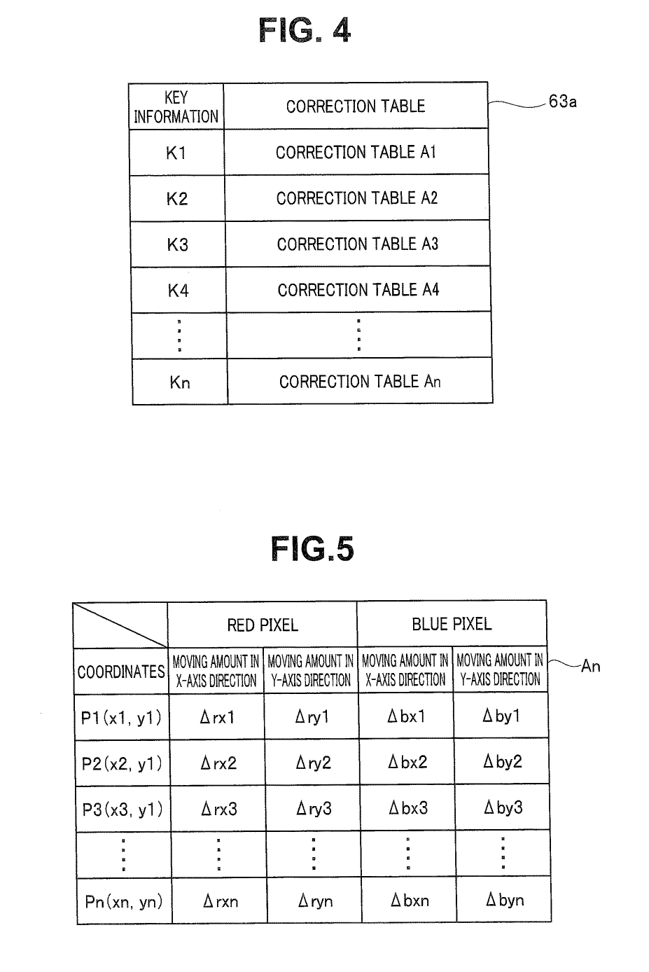

FIG. 4 is a chart showing an example of an association table 63a of the endoscope apparatus 1 according to the embodiment of the present invention. In the example shown in FIG. 4, the association table 63a includes n pieces of key information Kn and n number of correction tables An. Hereinafter just referred to as the key information Kn or the correction table An, when any one piece of or all pieces of the key information is referred to or any one of or all of the correction tables is referred to.

The control section 61 is configured to be capable of controlling operations of the respective sections or portions in the endoscope apparatus 1. The control section 61 includes a central processing unit (hereinafter, referred to as “CPU”) 62, a processor memory 63 that includes a volatile memory and a nonvolatile memory, a correction information acquisition portion 64, an image generation portion 65, and an image correction portion 66. The functions of the processing portions in the control section 61 are implemented by executing various kinds of programs stored in the processor memory 63 by the CPU 62.

The processor memory 63 stores a program for the processing portion that performs the key information setting processing to be described later, the association table 63a, a plurality of correction tables An, and a mapping table 63b, in addition to the programs for controlling the operations of the respective sections and portions in the endoscope apparatus 1.

In the association table 63a, the key information Kn and the correction table An are associated with each other, as shown in FIG. 4. The configuration of the correction table An will be described later.

The mapping table 63b includes information on the pixel positions of a raster-format image corresponding to the detection signal such that the detection signal inputted from the detection unit 41 can be converted into a raster-format picked-up image by mapping processing.

The correction information acquisition portion 64 is a circuit that acquires correction information according to the magnification chromatic aberration of the endoscope 3 from the scope memory 34 in the endoscope 3. The correction information acquisition portion 64 acquires the key information Kn from the scope memory 34, to output the acquired key information Kn to the image correction portion 66.

That is, the correction information includes the key information Kn associated with a predetermined correction table of the plurality of correction tables.

The image generation portion 65 is a circuit that generates a picked-up image of the subject, an image of which is picked up by the endoscope 3. The image generation portion 65 generates the picked-up image based on the image pickup signal acquired from the detection unit 41. More specifically, the image generation portion 65 performs, based on the mapping table 63b, mapping processing on the red, green and blue image pickup signals that are acquired along the spiral-shaped scanning path, and generates a raster-format picked-up image including a red image, a green image, and a blue image, to output the generated picked-up image to the image correction portion 66.

The image correction portion 66 is a circuit that corrects the magnification chromatic aberration in the picked-up image based on the key information Kn as the correction information. The image correction portion 66 extracts a predetermined correction table associated with the key information Kn from the plurality of correction tables An, based on the key information Kn, and corrects the magnification chromatic aberration in the picked-up image based on the extracted predetermined correction table. The image correction portion 66 outputs the corrected picked-up image to the display section 4.

### (Configuration of Correction Table An)

The configuration of the correction table An of the endoscope apparatus 1 will be described.

FIG. 5 is a chart showing an example of the correction table An of the endoscope apparatus 1 according to the embodiment of the present invention. In the example in FIG. 5, the correction table An includes n pieces of coordinate information Pn and n pieces of moving amount information Δrxn, Δryn, Δbxn, and Δbyn. Hereinafter just referred to as the coordinate information Pn or the moving amount information Δrxn, Δryn, Δbxn, and Δbyn, when any one piece of or all pieces of coordinate information or any one piece of or all pieces of moving amount information are referred to.

The correction table An shown in FIG. 5 includes information for correcting the magnification chromatic aberration in the picked-up image. The number of the correction tables An is set to n in advance in accordance with the attaching positions and directions of the optical system 33a and the n correction tables are stored in the processor memory 63. That is, the processor memory 63 includes a plurality of correction tables An according to the attaching positions and directions of the optical system 33a.

In the wavelength components of normal light, a G value indicating green color in the RGB color space approximates to a Y value indicating the illuminance value in the YCbCr color space. Therefore, the correction table An includes information for performing correction for matching the red image and the blue image with the green image such that the picked-up image approximates to the image in which only the CbCr value as the color difference component in the YCbCr color space is corrected.

Specifically, the correction table An includes the moving amount information Δrxn, Δryn, Δbxn, and Δbyn of the pixels. In FIG. 5, in the coordinate information Pn (xn, yn), for example, the moving amount of the red pixels in the X-axis direction is Δrxn, the moving amount of the red pixels in the Y-axis direction is Δryn, the moving amount of the blue pixels in the X-axis direction is Δbxn, and the moving amount of the blue pixels in the Y-axis direction is Δbyn.

Note that the correction tables An include the information for correcting the red image and the blue image in the embodiment, but may include information for correcting the images of other colors. For example, the colors of the images to be corrected may be red and green, or blue and green, or may be red, green, and blue.

Next, description will be made on the correction tables A1, A2, A3, and A4 according to the attaching positions and directions of the optical system 33a. For descriptive purpose, the correction tables A1, A2, A3, and A4 include moving amount information of bar patterns B1, B2, B3, and B4 in the picked-up image obtained by picking up the image of a measurement chart C, but the correction tables may be configured by the moving amount information Δrxn, Δryn, Δbxn, and Δbyn of the pixels.

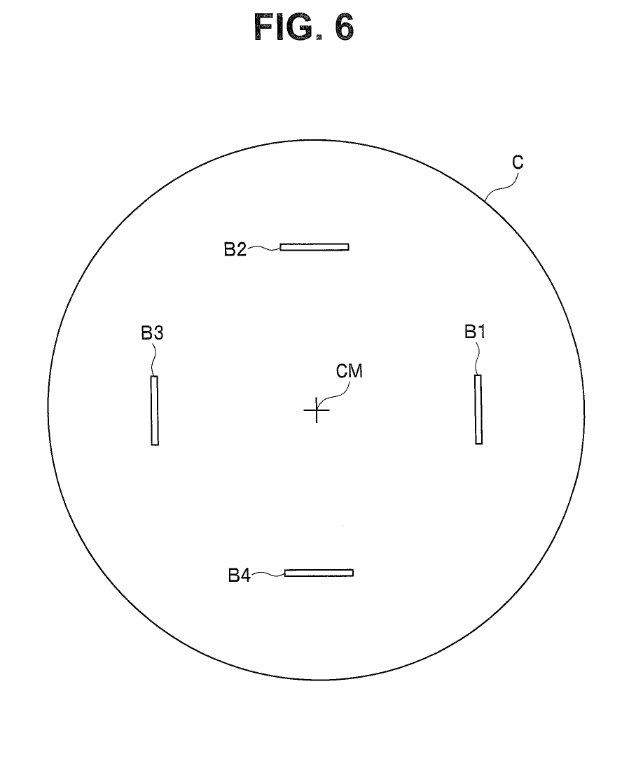

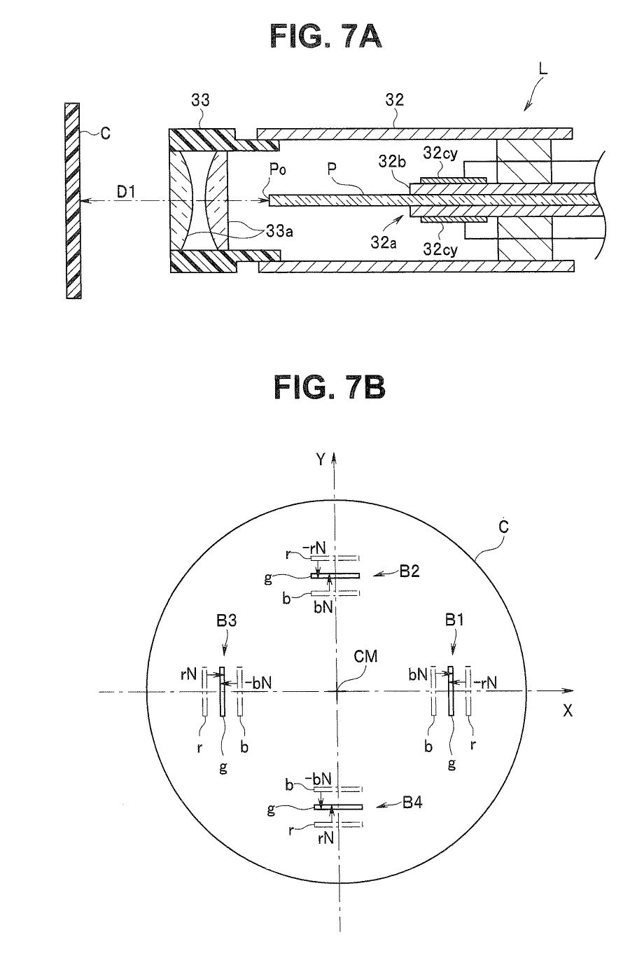

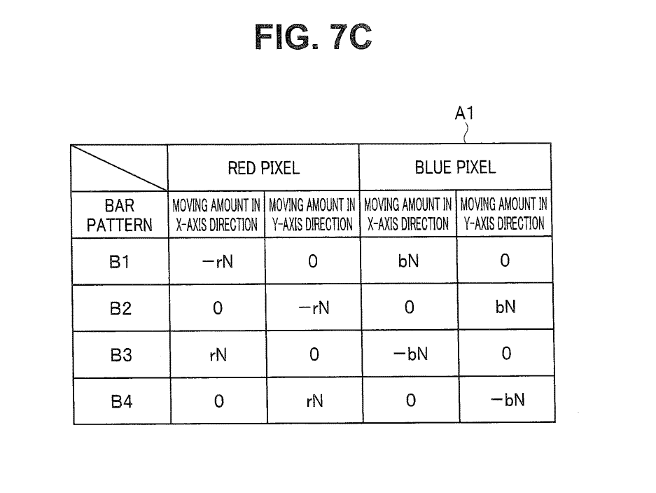

FIG. 6 illustrates an example of the measurement chart C of the endoscope apparatus 1 according to the embodiment of the present invention. FIG. 7A is an explanatory diagram for describing an exemplary configuration of the illumination portion L of the endoscope apparatus 1 according to the embodiment of the present invention. FIG. 7B is an explanatory diagram for describing the magnification chromatic aberration in the picked-up image obtained by the endoscope apparatus 1 according to the embodiment of the present invention. FIG. 7C is a chart showing an example of a correction table Al of the endoscope apparatus 1 according to the embodiment of the present invention.

First, description will be made on the measurement chart C. As shown in FIG. 6, the measurement chart C includes a center marker CM and the bar patterns B1, B2, B3, and B4 that are arranged respectively in four directions, with the center marker CM as the center. The base color of the measurement chart C is black, and the colors of the center marker CM and the bar pattersn B1, B2, B3, and B4 are white. The black base color is not shown in FIG. 6. Note that the bar pattersn B1, B2, B3, and B4 are shown as one bar pattern B1, one bar pattern B2, one bar pattern B3, and one bar pattern B4, respectively, in FIG. 6, for descriptive purpose. However, each of the bar patterns B1, B2, B3, and B4 may include three bar patterns arranged in the radial direction.

As shown in FIG. 7A, when the optical system 33a is attached at a predetermined attaching position and in a predetermined direction, the measurement chart C is arranged so as be apart from the emission end Po by a predetermined distance D1.

When the illumination light is emitted from the emission end Po, the illumination light is refracted at different angles depending on the color components included therein due to the magnification chromatic aberration of the optical system 33a, and applied to the measurement chart C. The return light from the measurement chart C is received by the light-receiving portion Ri, converted into the image pickup signal by the detection unit 41, to be inputted to the image generation portion 65. The image generation portion 65 refers to the mapping table 63b, generates a raster-format picked-up image based on the image pickup signal, and outputs the generated picked-up image to the image correction portion 66. The picked-up image inputted to the image correction portion 66 includes blue, green, and red images in which color shift occurs due to the magnification chromatic aberration of the optical system 33a, and whose sizes are different from one another. In FIG. 7B, for example, a blue bar pattern b, a green bar pattern g, and a red bar pattern r are arranged in sequence in the radial direction.

FIG. 7C shows an example of the correction table Al that is used when the optical system 33a is attached at a predetermined attaching position and in a predetermined direction. The correction table A1 includes the moving amounts of the red bar pattern r, and the blue bar pattern b. For example, in each of the bar patterns B1, B2, B3, and B4 in FIG. 7B, if the red bar pattern r moves in the direction of the center marker CM by a distance rN and the blue bar pattern b moves in the outside direction by a distance bN based on the correction table A1, the red bar pattern r and the blue bar pattern b are arranged at the same position as that of the green bar pattern g. When the red bar pattern r and the blue bar pattern b are arranged at the same position as that of the green bar pattern g, the color shift is eliminated, and the magnification chromatic aberration is corrected.

That is, the correction table A1 includes information for moving the red bar pattern r in the direction of the center marker CM by the distance rN and moving the blue bar pattern b in the outside direction by the distance bN.

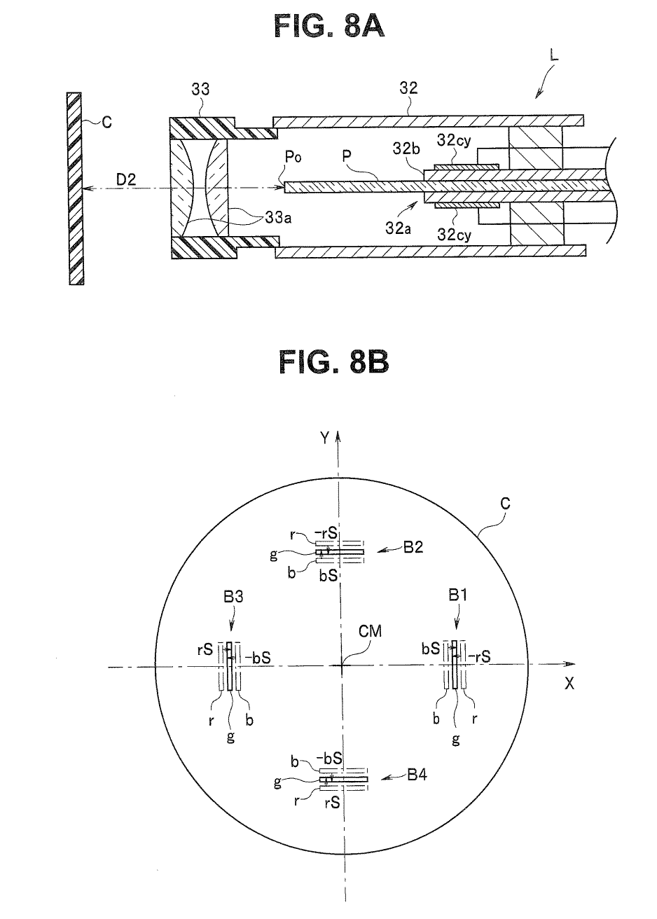

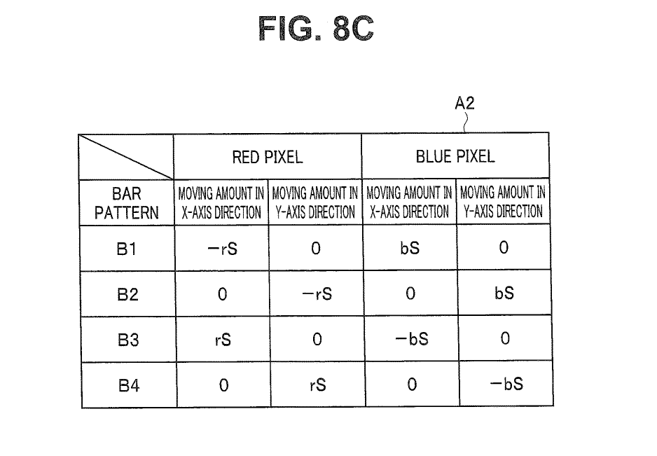

FIG. 8A is an explanatory diagram for describing an exemplary configuration of the illumination portion L of the endoscope apparatus 1 according to the embodiment of the present invention. FIG. 8B is an explanatory diagram for describing the magnification chromatic aberration in the picked-up image obtained by the endoscope apparatus 1 according to the embodiment of the present invention. FIG. 8C is a chart showing an example of the correction table A2 of the endoscope apparatus 1 according to the embodiment of the present invention.

As shown in FIG. 8A, if the optical system 33a is attached shifted in the distal end direction, the measurement chart C is arranged apart from the emission end Po by a predetermined distance D2 longer than the predetermined distance D1.

When the measurement chart C is apart from the emission end Po by the predetermined distance D2, color shift, which is smaller than the color shift in the case where the measurement chart C is apart from the emission end Po by the predetermined distance D1, occurs in the picked-up image.

As shown in FIG. 8B, for example, the red bar pattern r is shifted from the green bar pattern g in the outside direction by a distance rS shorter than a distance rN, and the blue bar pattern b is shifted from the green bar pattern g in the direction of the center marker CM by a distance bS shorter than a distance bN in the picked-up image.

FIG. 8C shows an example of the correction table A2 that is used when the optical system 33a is attached shifted in the distal end direction with respect to the predetermined attaching position and direction. The correction table A2 includes information for moving the red bar pattern r in the direction of the center marker CM by the distance rS and moving the blue bar pattern b in the outside direction by the distance bS. In other words, the correction table A2 includes information on the correction amount smaller than the correction amount of the magnification chromatic aberration in the correction table A1.

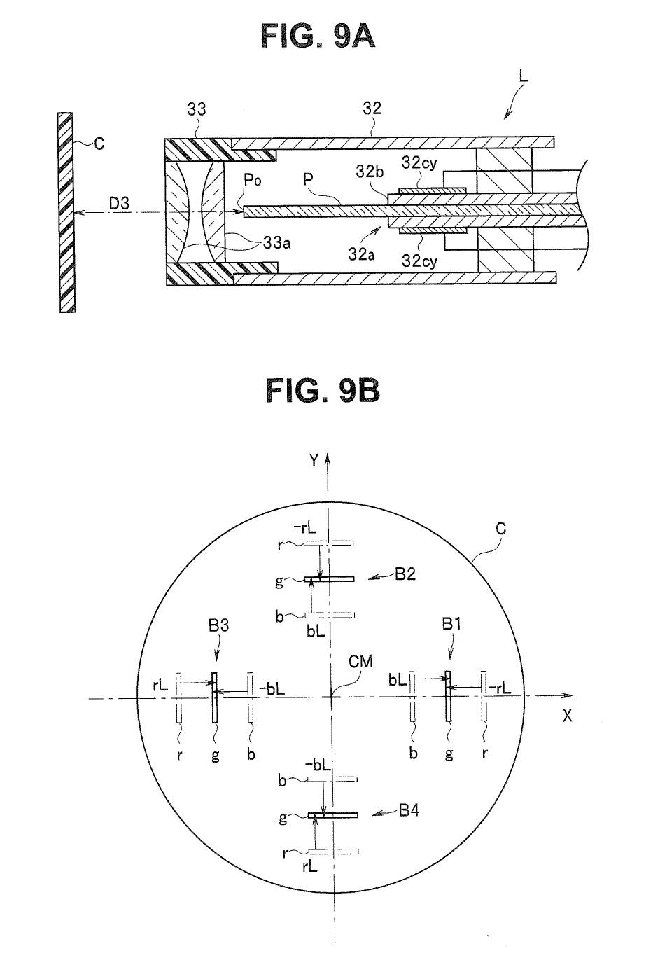

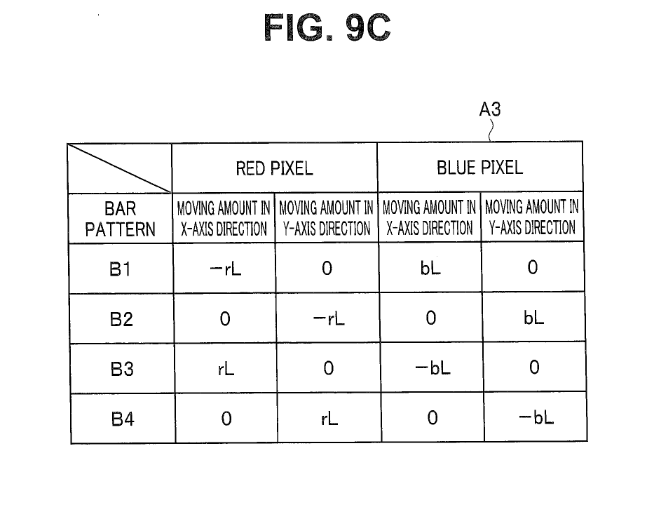

FIG. 9A is an explanatory diagram for describing an exemplary configuration of the illumination portion L of the endoscope apparatus 1 according to the embodiment of the present invention. FIG. 9B is an explanatory diagram for describing the magnification chromatic aberration in the picked-up image obtained by the endoscope apparatus 1 according to the embodiment of the present invention. FIG. 9C is a chart showing an example of a correction table A3 of the endoscope apparatus 1 according to the embodiment of the present invention.

As shown in FIG. 9A, if the optical system 33a is attached shifted in the proximal end direction, the measurement chart C is arranged apart from the emission end Po by a predetermined distance D3 shorter than the predetermined distance D1.

When the measurement chart C is apart from the emission end Po by the predetermined distance D3, the color shift, which is larger than the color shift in the case where the measurement chart C is apart from the emission end Po by the predetermined distance D1, occurs in the picked-up image.

As shown in FIG. 9B, for example, the red bar pattern r is shifted from the green bar pattern g in the outside direction by the distance rL longer than the distance rN and the blue bar pattern b is shifted from the green bar pattern g in the direction of the center marker CM by the distance bL longer than the distance bN in the picked-up image.

FIG. 9C shows an example of a correction table A3 that is used in the case where the optical system 33a is attached shifted in the proximal end direction with respect to the predetermined attaching position and direction. The correction table A3 includes information for moving the red bar pattern r in the direction of the center marker CM by the distance rL and moving the blue bar pattern b in the outside direction by the distance bL. In other words, the correction table A3 includes information on the correction amount larger than the correction amount of the magnification chromatic aberration in the correction table A1.

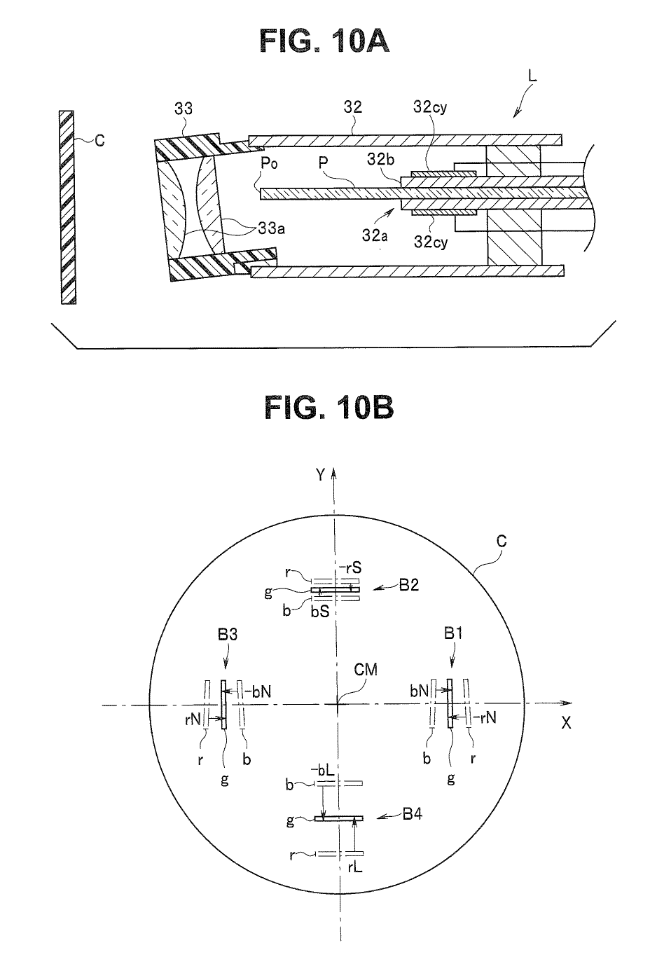

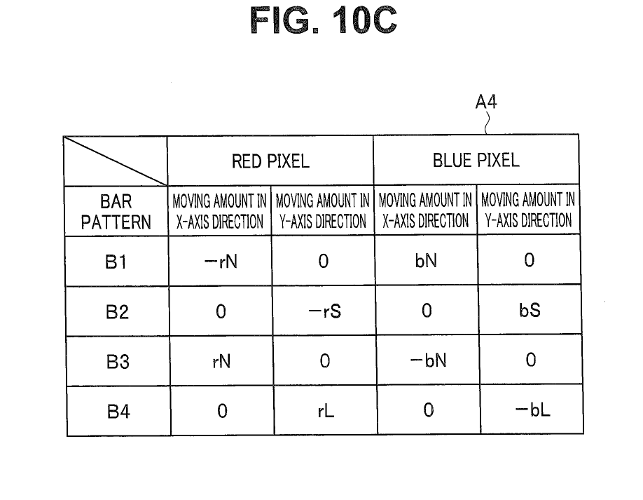

FIG. 10A is an explanatory diagram for describing an exemplary configuration of the illumination portion L of the endoscope apparatus 1 according to the embodiment of the present invention. FIG. 10B is an explanatory diagram for describing the magnification chromatic aberration in the picked-up image obtained by the endoscope apparatus 1 according to the embodiment of the present invention. FIG. 10C is a chart showing an example of a correction table A4 of the endoscope apparatus 1 according to the embodiment of the present invention.

As shown in FIG. 10A, when the optical system 33a is inclined with respect to the predetermined attaching position and direction, regions each having different magnification chromatic aberration are generated on a virtual circle that is apart from the center marker CM by a predetermined radius in the picked-up image.

As shown in FIG. 10B, for example, in the bar pattern B2 in the picked-up image, the magnification chromatic aberration, which is smaller than that in the bar pattern B4, occurs. Therefore, in the bar pattern B2, the red bar pattern r and the blue bar pattern b are shifted from the green bar pattern g by the distance rS and by the distance bS, respectively. On the other hand, in the bar pattern B4, the red bar pattern r and the blue bar pattern b are shifted from the green bar pattern g by the distance rL and by the distance bL, respectively.

FIG. 10C shows an example of the correction table A4 that is used in the case where the optical system 33a is attached inclined with respect to the predetermined attaching position and direction. The correction table A4 includes information on the correction amount of the magnification chromatic aberration, the correction amount gradually increasing from the region where the bar pattern B2 is arranged toward the direction of the region where the bar pattern B4 is arranged.

### (Operation)

### (Key Information Setting Processing)

Next, description will be made on the key information setting processing.

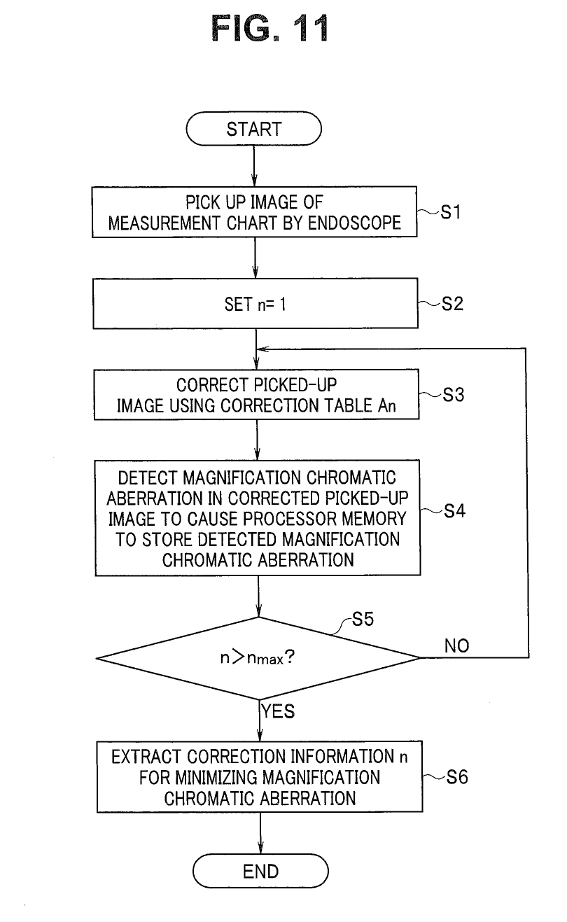

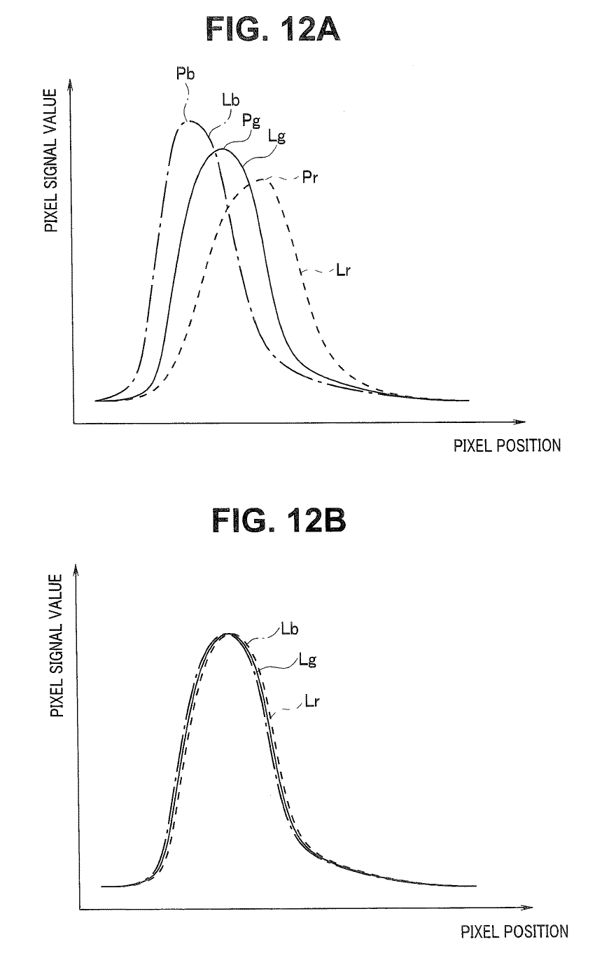

FIG. 11 is a flowchart showing an example of a flow of the key information setting processing of the endoscope apparatus 1 according to the embodiment of the present invention. FIGS. 12A and 12B are graphs illustrating a relation between a pixel position and a signal level in the picked-up image obtained by the endoscope apparatus 1 according to the embodiment of the present invention.

FIG. 11 shows that the key information setting processing is performed by the endoscope apparatus 1. However, the key information setting processing may be performed by a key information setting apparatus, not shown, which is configured to perform only the key information setting processing. The key information setting processing is performed by the control section 61 in FIG. 11, but may be performed manually.

The key information setting processing is processing for storing the key information Kn in the scope memory 34, which is performed before the factory shipment.

An image of the measurement chart C is picked up by the endoscope 3 (S1). The user arranges the measurement chart C on a surface perpendicular to the central axis of the protection pipe 32, and places the center marker CM on the central axis of the protection pipe 32. The endoscope 3 picks up the image of the measurement chart C. When the image of the measurement chart C is picked up, the control section 61 generates a picked-up image of the measurement chart C, the picked-up image including red, green, and blue images.

Counter information n is set to 1 (S2).

The picked-up image is corrected based on the correction table An (S3). The control section 61 reads the correction table An corresponding to the counter information n from the processor memory 63, and corrects the picked-up image based on the read correction table An.

The control section 61 detects the magnification chromatic aberration in the corrected picked-up image and causes the processor memory 63 to store the detected magnification chromatic aberration (S4). The control section 61 detects pixel signal values corresponding to the pixel positions in the respective corrected red, green, and blue images. For example, in FIG. 12A, the X axis indicates the pixel position, the Y axis indicates the pixel signal, and with regard to the detection result of the bar pattern B1, the dashed line indicates a red pixel signal value Lr, the solid line indicates a green pixel signal value Lg, and the one-dot-chain line indicates a blue pixel signal value Lb. In FIG. 12A, the respective pixel signal values Lr, Lg, and Lb are shifted from each other in the X-axis direction due to the color shift. The control section 61 detects peak values Pr, Pg, and Pb in the respective pixel signal values Lr, Lg, and Lb, and calculates difference amounts among the respective detected peak values Pr, Pg, and Pb, by a predetermined calculation. The control section 61 associates the calculated difference amounts with the value of counter information n, as the value indicating the magnification chromatic aberration, to cause the processor memory 63 to store the value.

The control section 61 determines whether the value of the counter information n exceeds the number nmax of the correction table An (S5). When the control section 61 determines that the value of the counter information n exceeds the number nmax of the correction table An (S5: YES), the processing proceeds to S6. On the other hand, when the control section 61 determines that the value of the counter information n does not exceed the number nmax of the correction table An (S5: NO), the value of the counter information n is added by 1, and the processing returns to S3.

The counter information n for a correction table Anmin for minimizing the magnification chromatic aberration is extracted (S6). As shown in FIG. 12B, when the magnification chromatic aberration is small, the pixel signal values Lr, Lg, and Lb approximate to one another. The control section 61, in S4, reads the difference amounts and the counter information n stored in the processor memory 63, and extracts the counter information nmin associated with the minimum difference amount by a predetermined sort processing and the like. The control section 61 causes the scope memory 34 to store the key information Kn corresponding to the extracted counter information nmin.

That is, the key information Kn is set according to the attaching position and direction of the optical system 33a of the endoscope 3 such that the magnification chromatic aberration can be corrected based on the correction table Anmin for minimizing the magnification chromatic aberration.

The processing from the steps S1 to S6 constitutes the key information setting processing.

(Image correction processing)

Next, description will be made on the image correction processing in the endoscope apparatus 1.

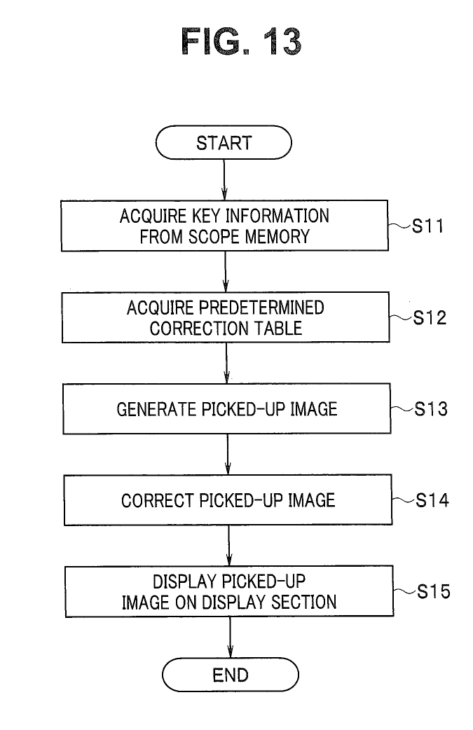

FIG. 13 is a flowchart showing an example of a flow of the image correction processing in the endoscope apparatus 1 according to the embodiment of the present invention.

The key information Kn is acquired from the scope memory 34 (S11). The correction information acquisition portion 64 acquires the key information Kn from the scope memory 34 and outputs the acquired key information Kn to the image correction portion 66.

A predetermined correction table is acquired (S12). The image correction portion 66 acquires from the processor memory 63 a predetermined correction table associated with the key information Kn acquired in S11.

A picked-up image is generated (S13). When the image of the subject is picked up by the endoscope 3, the image pickup signal is inputted to the image generation portion 65 through the detection unit 41. The image generation portion 65 generates a picked-up image based on the image pickup signal to output the generated picked-up image to the image correction portion 66.

The picked-up image is corrected (S14). The image correction portion 66 corrects the picked-up image acquired in S13, based on the predetermined correction table acquired in S12.

The picked-up image is outputted to the display section 4 (S15). The control section 61 outputs the picked-up image corrected in S14 to the display section 4.

The processing from the steps Sll to S15 constitutes the image correction processing.

That is, the endoscope processor 2 is capable of reading from the endoscope 3 the key information Kn set according to the attaching position and direction of the optical system 33a of the endoscope 3, acquiring a predetermined correction table associated with the key information Kn from the n number of correction tables An, and correcting the picked-up image.

According to the above-described embodiment, the endoscope processor 2 is capable of correcting the magnification chromatic aberration even in the case where the attaching position and direction of the optical system 33a are shifted from the predetermined attaching position and direction.

### MODIFIED EXAMPLE 1 OF THE EMBODIMENT

In the embodiment, the key information Kn is stored in the scope memory 34, the n number of correction tables An are stored in the processor memory 63, and a predetermined correction table is extracted from the n number of correction tables An. However, a correction table Ap may be stored in the scope memory 34 (see the two-dot-chain line in FIG. 1).

In the modified example 1 of the present embodiment, the correction table Ap is stored in the scope memory 34. The correction table Ap is extracted to be stored in the scope memory 34 before the factory shipment.

The correction information acquisition portion 64 outputs the correction table Ap acquired from the scope memory 34 to the image correction portion 66. The image correction portion 66 corrects the picked-up image based on the correction table Ap inputted from the correction information acquisition portion 64.

That is, the correction information includes the correction table Ap for correcting the magnification chromatic aberration in the picked-up image, and the image correction portion 66 corrects the magnification chromatic aberration in the picked-up image based on the correction table Ap acquired from the scope memory 34.

Such a configuration suppresses the storage amount of the processor memory 63 and enables the magnification chromatic aberration to be corrected even in the case where the attaching position and direction of the optical system 33a are shifted from the predetermined attaching position and direction.

### MODIFIED EXAMPLE 2 OF THE EMBODIMENT

In the embodiment, the image generation portion 65 generates the picked-up image based on the mapping table 63b, and the image correction portion 66 corrects the picked-up image based on the predetermined correction table. However, a correction image generation table 63c including both the information on the mapping table 63b and the information on the correction table An may be stored in the processor memory 63, and the image generation portion 65 may generate and correct the picked-up image based on the correction image generation table 63c.

That is, the correction image generation table 63c for generating a raster-format picked-up image based on the image pickup signal acquired along the spiral-shaped scanning path and correcting the magnification chromatic aberration in the picked-up image is stored in the processor memory 63, and the image generation portion 65 generates, based on the correction image generation table 63c, the picked-up image in which the magnification chromatic aberration is corrected, to output the generated picked-up image to the display section 4 (see two-dot-chain line in FIG. 1).

Such a configuration enables the correction table An and the mapping table 63b to be united as one correction image generation table 63c, and enables the function of the image correction portion 66 to be achieved with the generation of the picked-up image in the image generation portion 65, As a result, the storage amount of the processor memory 63 can be suppressed, and the magnification chromatic aberration can be corrected even in the case where the attaching position and direction of the optical system 33a are shifted from the predetermined attaching position and direction.

### MODIFIED EXAMPLE 3 OF THE EMBODIMENT

In the embodiment, the key information Kn is stored in the scope memory 34. However, the key information Kn and correction amount information Kn1 may be stored in the scope memory 34 as correction information (see the two-dot-chain line in FIG. 1).

The correction information acquisition portion 64 acquires the key information Kn and the correction amount information Kn1 from the scope memory 34 to output the acquired information to the image correction portion 66.

The image correction portion 66 extracts the correction table An from the processor memory 63 based on the key information Kn inputted from the correction information acquisition portion 64, determines a correction amount for the correction table An by a predetermined calculation based on the correction amount information Kn1, and corrects the picked-up image inputted from the image generation portion 65 by the determined correction amount based on the predetermined correction table.

That is, the correction information includes the key information Kn and the correction amount information Kn1, and the image correction portion 66 extracts the predetermined correction table associated with the key information Kn from the plurality of correction tables An, and corrects the magnification chromatic aberration in the picked-up image by the amount corresponding to the correction amount information Kn1, based on the predetermined correction table.

According to such a configuration, even in the case where the attaching position and direction of the optical system 33a are shifted from the predetermined attaching position and direction, the image correction is performed based on the key information Kn and the correction amount information Kn1, to thereby enable the magnification chromatic aberration to be corrected with a higher precision.

Note that the endoscope apparatus 1 is a scanning endoscope apparatus in the embodiment and the modified examples, but not limited to the scanning endoscope apparatus. The endoscope apparatus 1 may be the one including an image pickup section configured by CMOS, CCD, or the like.

### (Method of Color Correction)

The color-difference matrix method can be considered as the color correction method.

When the color correction is performed by using the color-difference matrix method, a problem of contrast deterioration occurs in the picked-up image due to the mixture of the red image and green image with low contrast into the blue image with high contrast when the image of blood vessels or the like is picked up.

In order to solve such a problem, color correction using the linear matrix method is performed on the picked-up image in the RGB color space, to transform the RGB color space into the YCbCr color space. Then, color correction is performed using the color-difference matrix method in the YCbCr color space, to transform the YCbCr color space into the RGB color space.

According to such color correction, coarse color adjustment is performed by applying gain only to the RGB colors with the linear matrix method, and then fine color adjustment is performed with the color-difference matrix method, to suppress the contrast deterioration in the picked-up image.

The respective “sections” and “portions” in the specification are conceptions corresponding to the respective functions in the embodiment and do not correspond one by one to a specific hardware or software. Therefore, in the specification, description has been made supposing virtual circuit blocks (sections, portions) including the respective functions in the embodiment. Further, the respective steps in the procedures in the present embodiment may be executed in different orders, a plurality of steps may be simultaneously executed, or the respective steps may be executed in a different order for each execution, unless contrary to the nature thereof Furthermore, all of or a part of the respective steps in the procedures in the present embodiment may be executed by a hardware.

The present invention is not limited to the above-described embodiment, and various changes, modifications, and the like are possible without departing from the gist of the present invention.

What is claimed is:

1. An endoscope processor comprising:
an image generation portion that generates a picked-up image of a subject, an image of which is picked up by an endoscope;
a correction information acquisition portion that acquires correction information corresponding to magnification chromatic aberration of the endoscope from a scope memory in the endoscope; and
an image correction portion that corrects the magnification chromatic aberration in the picked-up image based on the correction information.

2. The endoscope processor according to claim 1, further comprising:
a processor memory that stores a plurality of correction tables;
wherein the correction information includes key information associated with a predetermined correction table of the plurality of correction tables, and
the image correction portion extracts, based on the key information, the predetermined correction table associated with the key information from the plurality of correction tables, and corrects the magnification chromatic aberration in the picked-up image based on the extracted predetermined correction table.

3. The endoscope processor according to claim 2, wherein each correction table of the plurality of correction tables includes information for correcting the magnification chromatic aberration in the picked-up image.

4. The endoscope processor according to claim 3, wherein the each correction table is set according to an attaching position and direction of an optical system of the endoscope.

5. The endoscope processor according to claim 3, wherein the each correction table includes information for performing correction for matching a red image and a blue image with a green image.

6. The endoscope processor according to claim 1, further comprising
a processor memory that stores a plurality of correction tables,
wherein the correction information includes a predetermined correction table for correcting the magnification chromatic aberration in the picked-up image, and
the image correction portion corrects the magnification chromatic aberration in the picked-up image based on the predetermined correction table acquired from the scope memory.

7. The endoscope processor according to claim 1, wherein
the endoscope is a scanning endoscope,
the endoscope processor further comprises a processor memory that stores a correction image generation table for generating the picked-up image in a raster format based on an image pickup signal acquired along a spiral-shaped scanning path and correcting the magnification chromatic aberration in the picked-up image, and
the image generation portion generates the picked-up image in which the magnification chromatic aberration is corrected, based on the correction image generation table.

8. The endoscope processor according to claim 1, further comprising
a processor memory that stores a plurality of correction tables,
wherein the correction information includes key information and correction amount information, and
the image correction portion extracts, based on the key information, a predetermined correction table associated with the key information from the plurality of correction tables, and corrects the magnification chromatic aberration in the picked-up image by an amount corresponding to the correction amount information based on the correction table.
